SEQUENCE LISTING (9 sequences)

SEQ ID NO: 1 - protein, 40 aa, Unknown Organism
  Description of Unknown Organism Unknown A40 Peptide
DAEFRHDSGYEVHHQKLVFFAEDVGSNKGAIIGLMVGGVV

SEQ ID NO: 2 - protein, 42 aa, Unknown Organism [sequence omitted]

SEQ ID NO: 3 - protein, 37 aa, Homo sapiens [sequence omitted]

SEQ ID NO: 4 - protein, 21 aa, Homo sapiens
KTNMKHMAGAAAAGAVVGGLG

SEQ ID NO: 5 - protein, 44 aa, Artificial Sequence
  Description of Artificial Sequence Synthetic Peptide
KKQQQQQQQQQQQQQQQQQQQQQQQQQQQQQQQQQQQQQQQQKK

SEQ ID NO: 6 - protein, 147 aa, Homo sapiens
MKALIVLGLVLLSVTVQGKVFERCELARTLKRLGMDGYRGSLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 7 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 8 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 9 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2011/0020237 A1
(43) Pub. Date: Jan. 27, 2011

(54) Compositions and Methods for Inhibiting Drusen Formation and for Diagnosing or Treating Drusen-Related Disorders

(76) Inventors: Charles G. Glabe, Irvine, CA (US); Rakez Kayed, Irvine, TX (US); Ralf Langen, Pasadena, CA (US); Jeannie Chen, Pasadena, CA (US); Jose Mario Isas, Mission Viejo, CA (US); Volker Luibl, Alhambra, CA (US)

Correspondence Address: STOUT, UXA, BUYAN & MULLINS LLP, 4 VENTURE, SUITE 300, IRVINE, CA, 92618, US

(21) Appl. No.: 11/795,373

(22) PCT Filed: Jan. 17, 2006
(86) PCT No.: PCT/US06/01478

§ 371 Date: May 19, 2008

Related U.S. Application Data
(60) Provisional application No. 60/644,380, filed on Jan. 14, 2005.

(51) Int. Cl. A61K 49/04; A61K 39/395; A61P 27/02; A61P 25/28; C07K 16/18; A61P 27/06

(52) U.S. Cl. 424/9.4; 424/139.1; 530/387.9; 530/387.3

(57) ABSTRACT

Compositions of matter and methods for inhibiting drusen or drusen-like deposits and/or for treating diseases related to drusen or drusen-like deposits in human or animal subjects by administering to the subject a therapeutically effective amount of i) a conformational epitope of an aggregate that contributes to the formation or biosynthesis of drusen or drusen-like deposits and/or ii) an antibody that binds to a conformational epitope of an aggregate that contributes to the formation or biosynthesis of drusen or the drusen-like deposit.

### RELATED APPLICATIONS

This utility patent application claims priority to U.S. Provisional Patent Application Ser. No. 60/644,380 filed on Jan. 14, 2005, the entirety of which is expressly incorporated herein by reference.

This application is also a continuation in part of a) copending U.S. patent application Ser. No. 10/527,678 filed on Mar. 11, 2005, which is a Section 371 national stage application of PCT International Application No. PCT/US2003/28829 filed Sep. 12, 2003 which claims priority to U.S. Provisional Patent Application No. 60/410,069 filed Sep. 12, 2002 and b) copending United States PCT International Application No. PCT/US2004/029946 filed Sep. 12, 2004 which claims priority to U.S. Provisional Application 60/502,326 filed Sep. 12, 2003, the entireties of all such related applications being expressly incorporated herein by reference.

### FIELD OF THE INVENTION

This invention relates generally to compositions of matter and methods for medical treatment. More particularly, this invention relates to compositions and methods for inhibiting drusen or drusen-like deposits and/or for treating diseases related to drusen or drusen-like deposits.

### BACKGROUND OF THE INVENTION

The formation of insoluble extracellular deposits consisting of misfolded, aggregated protein is a hallmark of many neurodegenerative diseases. Notably, protein misfolding and aggregation are thought to underlie the pathogenesis of many amyloid diseases, such as Alzheimer and Parkinson diseases, whereby a stepwise protein misfolding process begins with the conversion of soluble protein monomers to pre-fibrillar oligomers, and progresses to insoluble amyloid fibrils.

Extracellular deposits known as “drusen” have been known to accumulate within the eyes of human beings as they age. Drusen can be observed directly under funduscopic examination and may be classified as either soft drusen or hard drusen, depending on relative size, abundance, and shape. Drusen typically forms beneath the basement membrane of the retinal pigmented epithelium (RPE) and the inner collagenous layer of Bruch membrane. Excessive or confluent areas of drusen in the macula are associated with the development of chorioretinal disorders, such as age-related macular degeneration (AMD).

Drusen have been shown to contain a number of immunomodulatory substances. It is believed that, in the pathogenesis of age-related macular degeneration, drusen formation results from a series of local inflammatory events on or near the macula. In some respects, these local inflammatory events are similar to local inflammatory events in brain tissue that have been associated with the development of Alzheimer's disease.

Recent characterizations of drusen have revealed protein components that are shared with amyloid deposits. However, characteristic amyloid fibrils have thus far not been identified in drusen. It has been hypothesized that non-fibrillar oligomers may be a common link in amyloid diseases. Oligomers consisting of distinct amyloidogenic proteins and peptides can be detected by a recently developed antibody that is thought to recognize a common structure. Significantly, oligomers exhibit cellular toxicity, which suggests that they play a role in the pathogenesis of neurodegenerative diseases.

Amyloid beta (A beta or Aβ), a peptide that plays a significant role in the pathogenesis of Alzheimer's disease, has also been found to be present in substructural vesicular components of drusen known as “amyloid vesicles.” Thus, it is theorized that Aβ deposition may play a significant roll in the local inflammatory events that contribute to atrophy of the retinal pigmented epithelium, drusen biogenesis, and the pathogenesis of chorioretinal disorders such as AMD as well as Alzheimer's disease.

Drusen, or deposits of material similar to drusen, may also be associated with other disorders. For example, current evidence suggests that there is a relationship between the presence of drusen and the development of elastosis. Elastosis is a term used to identify a group of conditions in which the elastic fibers in the skin undergo hyperplasia and/or rearrangement. While skin that has been exposed to sun over decades of life may be expected to exhibit signs of elastosis, the presence of elastotic lesions in the skin of sun protected areas of the body has been shown to correlate with the incidence of neovascular AMD.

Drusen-like deposits may also be involved in the pathogenesis of certain kidney diseases. Amorphous, electron-dense deposits near the kidney's basement membrane (and at some extrarenal sites such as the spleen) have been found in a subgroup of patients suffering from a kidney disorder known as membranoproliferative glomerulonephritis type II. Patients in whom these deposits form are said to have “dense deposit disease.” These deposits resemble drusen and patients with dense deposit disease frequently develop AMD. Kidney dense deposits do not a stain with Congo red and lack β-pleated fibrillar components. Thus, they differ from amyloids. However, like drusen, dense kidney deposits are immunoreactive with antibodies against vitronectin, immunoglobulin, and complement C5. Collectively, these data suggest that dense deposits are compositionally similar, although not identical, to drusen.

Also, The hylin deposits in idiopathic cardiomyopathy are similar to drusen in the sense that oligomers rather than fibrils tend to accumulate. This may also be the case in lens cataracts in the eye.

Recent data also suggests that drusen may be related to atherosclerosis. In this regard, drusen contain a number of constituents that are also contained in atherosclerotic plaques, including lipids, vitronectin, apolipoprotein E, calcium and complement components. Also, there is a recognized clinical correlation between the development of atherosclerosis of the carotid artery and advanced AMD. Other similarities between drusen and atherosclerotic plaques also exist. Thus, in at least some instances, there may be common pathogenic factors involved in the formation of drusen and atherosclerotic plaque.

The precise origin(s) of drusen-associated proteins remains to be resolved. Some drusen constituents (e.g., plasma proteins such as amyloid P component and prothrombin) may pass out of choroidal vessels and into the extracellular space adjacent to the RPE, where they might bind to one or more ligands associated with developing drusen. Other drusen constituents might be secreted by local retinal, RPE and/or choroidal cells.

### SUMMARY OF THE INVENTION

Applicants have determined that certain toxic amyloid-like oligomers are contained in drusen. The present invention provides methods for inhibiting the formation or biosynthesis of drusen (and possibly other drusen-like deposits) by blocking or inhibiting such toxic oligomers and/or for facilitating the break-down, degradation and/or clearance of drusen or drusen-like material.

In accordance with the invention, the toxic amyloid-like oligomers associated with drusen biosynthesis, formation and/or maintenance may be blocked or inhibited by administering to a human or animal subject, a therapeutically effective amount of a composition described herebelow and in incorporated U.S. patent application Ser. No. 10/527,678 (which is based on PCT International Patent Application No. PCT/US2003/028829 and published as WO 2004/024090). These compositions comprise one or more conformational epitopes found on amyloid peptide aggregates, and antibodies to such epitopes (e.g., anti-oligomer specific antibodies). The invention further includes antibodies which bind to these conformational epitopes as well as methods for making such antibodies and methods for the detection, treatment and prevention of diseases and/or identification of potential therapies (e.g., drug screening) using such antibodies.

Further in accordance with the invention, the toxic amyloid-like oligomers associated with drusen biosynthesis, formation and/or maintenance may be blocked or inhibited by administering to a human or animal subject, a therapeutically effective amount of a composition described herebelow and in copending PCT International Patent Application No. PCT/US2004/029946 (published as WO2005/025516), which is also expressly incorporated herein by reference. These compositions comprise polyclonal and monoclonal antibodies that are specific to conformational epitope(s) of aggregate(s) or oligomers (e.g., anti-oligomer specific antibodies) which contribute to amyloid fibril formation in human or animal subjects who suffer from amyloid disorders (e.g., drusen formation, age related macular degeneration, etc.) and the hybridomas and monoclonal antibodies produced therefrom. Also, the use of such antibodies in the passive immunization of human or animal subjects against amyloid diseases including Alzheimer's Disease, macular degeneration, other chorioretinal pathologies, and numerous others. The monoclonal antibodies may be administered concomitantly or in combination with anti-inflammatory agents, such as gold or gold containing compounds, to decrease neural inflammation associated with amyloid diseases (e.g., age related macular degeneration).

Still further in accordance with the invention, the methods of the present invention may be used to effect passive immunization against drusen and/or drusen related disorders by administering to a human or animal subject an anti-oligomer specific antibody which causes detoxification of amyloid oligomer(s) that participate in the biosynthesis or formation of drusen. Alternatively, the methods of the present invention may be used to effect active immunization through administration to a human or animal subject of specific antigen(s) that result in the formation of anti-oligomer specific antibodies that lead to clearance of toxic amyloid oligomers that participate in the biosynthesis or formation of drusen. These antigens, as described herebelow and in parent application Ser. No. 10/527,678 (based on PCT International Patent Application No. PCT/US2003/028829 and published as WO 2004/024090), elicit a specific immune response that results in the production of conformation-dependent antibodies that specifically recognize amyloid oligomers.

Still further in accordance with the invention, there is provided a method for inhibiting the formation and/or biosynthesis of, or for causing diminution of, drusen or a drusen-like deposit in a human or animal subject or for preventing or treating a disease or disorder that is associated with drusen or drusen-like deposits. Such method generally comprises the step of administering to the subject, in a therapeutically effective amount, a composition that comprises at least one of:
i) a conformational epitope of an aggregate that contributes to the formation or biosynthesis of drusen or drusen-like deposits; and
ii) an antibody that binds to a conformational epitope of an aggregate that contributes to the formation or biosynthesis of drusen or the drusen-like deposit.

In at least some applications of the invention, the aggregate may comprise from about 2 through about 20 subunits. In instances where a conformational epitope is administered to the subject, the composition may comprise a peptide and, in at least some embodiments, that peptide may comprise an amino acid sequence specified as SEQ ID NO. 1-9 described herebelow. In instances where an antibody is administered to the subject, the antibody may be polyclonal or monoclonal. A monoclonal antibody for this application may be generated by immunizing mice or other mammals with an antigen that is conformationally-constrained, as described herebelow. In some embodiments of the invention, the composition administered to the subject may be conformationally constrained in a shape that corresponds to a conformational-dependent epitope of an aggregate that contributes to the formation or biosynthesis of drusen or drusen-like deposits. Such conformational constraint may be achieved in any suitable way, such as by attaching or otherwise associating the composition with a surface of some matter having the desired shape. The surface may be on a film, particle, sheet, protein etc. In some instances, the composition may be constrained on the surface of a protein that comprises a β-pleated sheet. A β-pleated sheet is a secondary structure found in proteins in which hydrogen bonds are formed between two parts of the protein chain that can be far apart.

Still further in accordance with the invention, anti-oligomer specific antibodies may be used as drug delivery agents. In this regard, anti-oligomer specific antibodies may be crosslinked or otherwise bound to a drug or other therapeutic agent to facilitate targeted delivery of the drug or other therapeutic agent directly to drusen or to toxic oligomers involved in drusen biosynthesis or drusen formation.

Still further in accordance with the invention, anti-oligomer specific antibodies may be labeled with traceable labels (e.g., fluorophores) using techniques well known in the art. These labeled antibodies may be injected intravenously or otherwise administered such that the labeled antibodies will bind to toxic oligomers involved in drusen biosynthesis or drusen formation. Fluorescent angiography or other suitable techniques known in the art may then be used to visualize, locate, map and/or quantify any areas in the eye or elsewhere in the vasculature where those toxic oligomers are present, thereby determining locations at which drusen deposits are likely to develop and/or have already developed.

Still further in accordance with the invention, the toxic amyloid-like oligomers associated with drusen biosynthesis, formation and/or maintenance may be blocked or inhibited by administering to a human or animal subject, a therapeutically effective amount of an amyloid beta-derived diffusible ligand (ADDL) or an antibody that binds to ADDLs. Examples of such antibodies and ADDLs are known in the art and described in published United States Patent Application 2003/0068316, which is expressly incorporated herein by reference.

Still further in accordance with the invention, the toxic amyloid-like oligomers associated with drusen biosynthesis, formation and/or maintenance may be blocked or inhibited by administering to a human or animal subject, a therapeutically effective amount of an antibody that binds to an epitope within residues 1-7 of amyloid beta and/or a polypeptide that comprises an immunogenic fragment of amyloid beta and/or other compositions that inhibit the formation of amyloid beta as described in U.S. Pat. Nos. 6,787,637; 6,787,139; 6,787,138; 6,787,143; 6,787,144; 6,787,140; 6,787,523; 6,787,427; 6,750,324 and published United States Patent Application Nos. 2004/0175394; 2004/0171816; 2004/0171815; 2004/0170641 and 2004/0166119, which are expressly incorporated herein by reference.

Further aspects and elements of the invention will be understood upon reading of the detailed description and examples set forth herebelow.

### BRIEF DESCRIPTION OF THE DRAWINGS

FIGS. 1A-1H are confocal laser micrographs showing immunolocalization of amyloidogenic oligomers in drusen. Specifically, FIGS. 1A, 1C, 1E & 1G are differential interference contrast images; 1B, 1D, 1F & 1H are confocal fluorescence images of amyloid oligomer cores (green, FITC channel). As shown, drusen exhibit amyloid oligomer reactivity in the form of a core-like structure that accumulates centrally within drusen and in close proximity to Bruch membrane. Autofluorescence of lipofuscin granules in the RPE cytoplasm is imaged in red (Cy3 channel). (FIGS. 1A & 1B) Anti-oligomer-specific antibody recognizes a spherical structure (˜15 μm) in a small druse (˜30 μm). (FIGS. 1C-1F) Two larger drusen with centrally located core structure. (FIGS. 1G & 1H) A very large macular soft druse from an 81-year-old female donor. Despite the difference in sizes and shapes of the drusen, the amyloid oligomer cores remain 10-15 μm in size. RPE, retinal pigmented epithelium; Bm, Bruch membrane. Bar=10 μm.

FIGS. 2A-2J show the presence of amyloid oligomers in drusen and thickened Bruch membrane. Amyloid oligomer reactivity is visualized with fluorescein (green), and lipofuscin autofluorescence is visualized using the Cy3 channel (red). Multiple amyloid oligomer cores are sometimes observed in large drusen (FIGS. 2A & 2B), as if a large druse may have formed from the fusion of several smaller drusen. The amyloid oligomer cores retain their size and their relative positions within the druse and in proximity to Bruch membrane. Within eyes that contain drusen, the oligomers occasionally accumulate above Bruch membrane, in the form of basal linear (FIGS. 2C & 2D) or basal laminar (FIGS. 2E-2H) deposits, particularly in instances where Bruch membrane appeared to be thickened. Staining within RPE cells was also observed (FIG. 2H). FIGS. 2C and 2D are differential interference contrast images of 2D and 2F, respectively. Specificity of the antibody in cryosections is demonstrated in adjacent sections of a large druse (FIGS. 2I & 2J). Multiple amyloid oligomer cores are visualized through use of the anti-oligomer antibody (FIG. 2I). Reactivity is eliminated when the primary antibody is pre-incubated with amyloid oligomers synthesized from the Aβ₁₋₄₀ peptide (FIG. 2J). RPE, retinal pigmented epithelium; Bm, Bruch membrane. Bar=10 μm.

FIGS. 3A-3B are ELISAs of retinal extracts using the anti-oligomer antibody. (FIG. 3A) Increasing amounts of oligomers made from the Aβ₁₋₄₀ peptide result in a dose-dependent response when incubated with the anti-oligomer-specific antibody (black circles). Little or no reactivity was observed when the Aβ₁₋₄₀ oligomers were incubated without the primary antibody (white circles). (FIG. 3B) Dose-dependent reactivity was observed when the anti-oligomer-specific antibody was incubated with increasing amounts of extract prepared from dissected Dr/RPE/Bm tissue from a 76-year-old male donor (black circles). Little or no reactivity was observed when the primary antibody was omitted (white circles). Extracts prepared from the neural retina (black triangles) of the same donor eye did not show a dose-dependent response when incubated with the anti-oligomer-specific antibody. Dr, drusen; RPE, retinal pigmented epithelium; Bm, Bruch membrane.

FIGS. 4A-4F show the morphology of amyloid oligomer cores in drusen at higher magnification. (FIGS. 4A-4C) Confocal micrographs of drusen. Amyloid oligomer cores are labeled with fluorescein (green), and lipofuscin autofluorescence in the RPE is visualized in red (Cy3 channel). Amyloid oligomer cores seem to consist of an aggregate of small vesicular structures (white arrowheads) that increase in density toward the center (FIG. 4A). Some of these vesicular structures appear to extend toward the RPE with diminishing density (B, arrowheads). Occasionally, the amyloid oligomer cores are seen to penetrate through Bruch membrane and extend toward the choroid (FIG. 4C, arrowhead). Ultrastructure of an amyloid oligomer core is depicted in an immunogold-labeled electron micrograph (FIG. 4D, inset), wherein gold particles decorate vesicular structures that are heterogeneous in size. The highest density of gold particles seen in D is from the region above Bruch membrane (rectangle, FIG. 4D). RPE, retinal pigmented epithelium; Bm, Bruch membrane; Ch, choroid. Bar (FIG. 4E)=2 μm. Bar (FIG. 4F)=100 nm. FIGS. 5A-5L show the co-distribution of amyloid oligomer cores and other known drusen components. DR. In all confocal images amyloid oligomer cores are labeled with fluorescein. HLA-DR is labeled with Texas Red (FIGS. 5B-5D). Both antigens are present in a large druse (FIG. 5A, differential interference contrast; FIG. 5B, confocal microscopy), wherein the amyloid oligomer core is enveloped within the HLA-DR reactive region. At higher magnification, it is clear that the amyloid oligomer core and HLA-DR reactive subdomain do not co-localize in these drusen. In one instance, the HLA-DR reactive region, perhaps reflecting a dendritic cell process, is observed as originating from the choroid, coming in close proximity to Bruch membrane, and contacting the condensation of vesicular structures that represent the amyloid oligomer core (FIG. 5C). In another instance, HLA-DR reactivity is observed as encompassing the choroid, Bruch membrane and the druse. Within the druse, HLA-DR reactivity appears to surround the oligomer core, with no indication of co-localization (FIG. 5D). Similarly, no co-localization was observed with vitronectin (FIGS. 5F-5H) or Aβ (FIGS. 5J-5L), both labeled with Texas Red. Lipofuscin autofluorescence within RPE is also visualized in the Cy3 channel. Dr, drusen; RPE, retinal pigmented epithelium; Bm, Bruch membrane. Bar=10 μm.

FIG. 6 shows that amyloid oligomers are toxic to cultured primary human RPE cells. Cell viability was assessed by MTT reduction. Increasing amounts of amyloid oligomers made from Aβ show a dose-dependent toxicity to cultured RPE cells. This toxicity is largely blocked by adding equal molars of the anti-oligomer antibody, Aβ. Error bars represent standard deviation, N=3.

### DETAILED DESCRIPTION AND EXAMPLES

### Definitions

As used in this patent application, the following terms shall have the following meanings:

The term “adjuvant” refers' to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

The term “amyloid beta,” [symbol] or [symbol] A beta peptide” refers to peptides which comprise low molecular weight soluble oligomers, prefibrillar aggregates, fibrils and amyloid deposits each associated with AD. Amyloid [symbol] beta peptides include, without limitation, [symbol] 39, [symbol] 40, [symbol] 41, [symbol] 42 and [symbol] 43 which are 39, 40, 41, 42 and 43 amino acid amino acids in length, respectively.

An “amyloid peptide” is a peptide that is present in amyloid forms including amyloid peptide intermediates, low molecular weight soluble oligomers, amyloid fibrils and amyloid plaques.

The term “antibody” is used to include intact antibodies and binding fragments thereof, including but not limited to, for example, full-length antibodies (e.g., an IgG antibody) or only an antigen binding portion (e.g., a Fab, F(ab′)₂ or scFv fragment). Typically, fragments compete with the intact antibody from which they were derived for specific binding to an antigen. Optionally, antibodies or binding fragments thereof, can be chemically conjugated to, or expressed as, fusion proteins with other proteins.

“Anti-oligomer antibody” or “Anti-oligomer” refers to an antibody that binds to amyloid peptide aggregate intermediates but does not bind to or does not specifically bind to amyloid peptide monomers, dimers, trimers or tetramers.

Compositions or methods “comprising” one or more recited elements may include other elements not specifically recited. For example, a composition that comprises an amyloid A beta peptide may encompass both an isolated amyloid A beta peptide as a component of a larger polypeptide sequence or as part of a composition which includes multiple elements.

The term “epitope” or “antigenic determinant” refers to a site on an antigen to which B and/or T cells respond or a site on a molecule against which an antibody will be produced and/or to which an antibody will bind. For example, an epitope can be recognized by an antibody defining the epitope.

A “linear epitope” is an epitope wherein an amino acid primary sequence comprises the epitope recognized. A linear epitope typically includes at least 3, and more usually, at least 5, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the antibody defining the epitope). Typically a conformational epitope comprises an increased number of amino acids relative to a linear epitope. With regard to recognition of conformational epitopes, the antibody recognizes a 3-dimensional structure of the peptide or protein. For example, when a protein molecule folds to form a three dimensional structure, certain amino acids and/or the polypeptide backbone forming the conformational epitope become juxtaposed enabling the antibody to recognize the epitope. Methods of determining conformation of epitopes include but are not limited to, for example, x-ray crystallography 2-dimensional nuclear magnetic resonance spectroscopy and site-directed spin labeling and electron paramagnetic resonance spectroscopy. See, for example, Epitope Mapping Protocols in Methods in Molecular Biology, Vol. 66, Glenn E. Morris, Ed. (1996), the disclosure of which is incorporated in its entirety herein by reference.

A “drusen-like” deposit or “drusen like” material, as referred to herein, is any extracellular protein deposit that contains, or whose biosynthesis involves, the production of an oligomer and wherein the biosynthesis, formation and/or maintenance of such oligomer may be blocked or inhibited by one or more conformational epitopes found on amyloid peptide aggregates and/or antibodies to such epitopes (e.g., anti-oligomer specific antibodies). These drusen-like deposits include but are not necessarily limited to electron-dense deposits near the kidney's basement membrane associated with membranoproliferative glomerulonephritis type II and dermal or skin deposits associated with elastosis.

The term “immunological response” or “immune response” relates to the development of a beneficial humoral (antibody mediated) and/or a cellular (mediated by antigen-specific T cells or their secretion products) response directed against an amyloid peptide in a recipient patient. Such a response can be an active response induced by administration of monoclonal antibody or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity.

An “immunogen” is capable of inducing an immunological response against itself upon administration to a subject, optionally in conjunction with an adjuvant.

“Isolated” means purified, substantially purified or partially purified. Isolated can also mean present in an environment other than a naturally occurring environment. For example, an antibody that is not present in the whole blood serum in which the antibody would ordinarily be found when naturally occurring is an isolated antibody.

“Low molecular weight aggregate”, “low molecular weight amyloid aggregate”, “low molecular weight oligomer” and “low molecular weight soluble oligomer” refer to amyloid peptides present in aggregates of less than four or five peptides. In one specific example, low molecular weight A refers to the low molecular weight soluble oligomers found associated with AD.

The term “patient” includes human and other animal subjects that receive therapeutic, preventative, experimental or diagnostic treatment or a human or animal (including subjects and/or research animal models) having a naturally occurring or experimentally induced disease or being predisposed to a disease.

“Prefibrillar aggregates”, “micellar aggregates”, “high molecular weight aggregation intermediates,” “high molecular weight amyloid peptide aggregates”, “high molecular weight soluble amyloid peptide aggregates” “amyloid peptide aggregates”, “soluble aggregate intermediates”, “amyloid oligomeric intermediates”, “oligomeric intermediates” and “oligomeric aggregates” or simply, “intermediates” refer to aggregations which include more than three individual peptide or protein monomers, for example, more than four peptide or protein monomers. The upper size of prefibrillar aggregates includes aggregations of oligomers which form spherical structures or micelles and stings of micelles which lead to fibril formation. The molecular weight of a prefibrillar aggregate may be in a range of about 10 kDa to about 100,000,000 KDa, for example, about 10 kDa to about 10,000,000 or 1,000,000 KDa. However, this size range is not intended to be limiting and prefibrillar aggregates are not defined by a molecular weight range.

“Annular protofibrils” are a particular subset of prefibrillar aggregates in which 3 to 10 spherical oligomer subunits are arranged in an annular or circular fashion with a hollow center that appears as a pore in electron or atomic force micrographs.

“Protofibrils” are prefibrillar aggregates which include spherical structures comprising amyloid [symbol] peptides that appear to represent strings of the spherical structures forming curvilinear structures.

“Specific binding” between two entities means an affinity of at least 10⁶, 10⁷, 10⁸, 10⁹ M⁻¹, or 10¹⁰ M⁻¹. Affinities greater than 10⁸ M⁻¹ are preferred for specific binding.

The term “substantial identity” means that two peptide sequences, when optimally aligned, such as by the programs GAP or BESTFIT using default gap weights, share at least 65 percent sequence identity, for example, at least 80 percent or 90 percent sequence identity, or at least 95 percent sequence identity or more, for example, 99 percent sequence identity or higher.

Preferably, residue positions in an alignment which are not identical differ by conservative amino acid substitutions, i.e., substitution of an amino acid for another amino acid of the same class or group. Some amino acids may be grouped as follows: Group I (hydrophobic side chains): leu, met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe. Non-conservative substitutions may include exchanging a member of one of these classes for a member of another class.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm may then be used to calculate the percent sequence identity for the test sequence (s) relative to the reference sequence, based on the designated program parameters. Optimal alignment of sequences for comparison can be conducted, for example, by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix, see for example, Henikoff & Henikoff, Proc. Natl. Acad. Sci. USA 89, 10915 (1989). Conservative substitutions involve substitutions between amino acids in the same class.

A “therapeutic agent” or “therapeutic” is a substance useful for the treatment or prevention of a disease in a patient. Therapeutic agents of the invention are typically substantially pure. This means that an agent is typically at least about 50% w/w (weight/weight) pure, as well as being substantially free from proteins and contaminants which interfere with the efficacy of the therapeutic. The agents may be at least about 80% w/w and, more preferably at least 90% w/w or about 95% w/w in purity. However, using conventional protein purification techniques, homogeneous peptides of 99% w/w or more can be produced.

### Correlation Between AMD and Drusen:

Despite the well-established correlation between the presence of drusen and AMD, the underlying cause of drusen formation and its role in RPE and photoreceptor cell degeneration are not fully understood.

Recent evidence suggests that drusen formation and AMD share some similarities with amyloid diseases, such as Alzheimer disease (AD) and Parkinson disease (PD). Like AMD, amyloid diseases are strongly correlated with advancing age and the formation of deposits. Moreover, these amyloid deposits contain a wide range of lipids and proteins, many of which are also present in drusen. Shared components of amyloid deposits and drusen include proteins such as vitronectin, amyloid P, apolipoprotein E, and even the amyloid A beta (Aβ) peptide that is associated with amyloid plaques in Alzheimer disease. In humans, the APOE*4 allele shows a strong positive association with Alzheimer disease. Interestingly, expression of the APOE*4 allele in transgenic mice leads to ocular changes that mimic the pathology associated with human AMD. In addition, acute phase reactants, complement components, immune modulators, and other inflammatory mediators are present in amyloid deposits as well as in drusen, suggesting a possible common role for the inflammatory pathway in AMD and amyloid diseases. It is particularly noteworthy that the presence of complement components, such as C5, C5b9 and C3 fragments, had been observed in drusen of varying sizes and shapes, from small “hard” drusen to large “soft” drusen, in aging eyes as well as in AMD eyes. These observations are consistent with the idea that complement activation may be involved in drusen biogenesis. Together with the recent discovery that a polymorphism in complement factor H increases the risk factor of AMD, substantial attention is now focused on the role of inflammation in the pathogenesis of this disease.

Despite the shared similarities mentioned above, AMD has thus far not been classified as an amyloid disease. Among the principal differences is the fact that classical amyloid diseases typically exhibit large amounts of amyloid fibrils. For example, in the case of AD, the characteristic plaques consist primarily of fibrillar Alzheimer Aβ peptide, while the Lewy bodies found in PD are abundant in α-synuclein fibrils. These amyloid fibrils are elongated, 6 to 15 nm wide rod-like structures of indeterminate length that are characterized by a common cross p structure. In addition to their related structural features, amyloid fibrils display characteristic tinctorial properties, such as thioflavin T and congo red staining. Though drusen do stain with thioflavin T and congo red, the characteristic apple green birefringence often seen in congo red-stained amyloid fibrils is not present. Although amyloid proteins such as the Aβ peptide, transthyretin, immunoglobulin light chains, and amyloid A are found in drusen and sub-RPE deposits, electron microscopy studies have yielded sparse evidence of the presence of bona fide amyloid fibrils. These observations have precluded AMD from being viewed as a classical amyloid disease.

Amyloid fibril formation is a multi-step protein misfolding cascade of molecular events wherein a monomeric protein undergoes a conformational reorganization into a number of different oligomeric, β-sheet-containing structures that ultimately convert into amyloid fibrils. Numerous studies of various amyloid diseases have led to the perception that pre-fibrillar oligomers, rather than amyloid fibrils, might be the primary toxic agents. This notion has been supported by animal models demonstrating that amyloid fibrils do not seem to be required for the pathogenesis of amyloid diseases. These results suggest that additional diseases might be identified wherein pathogenic pre-fibrillar oligomers are present without significant accumulations of amyloid fibrils. Recent evidence suggests that desmin-related cardiomyopathy may be such a disease.

### The Presence of Prefibrillar Amyloid Oligomers in Drusen

Applicants carried out experiments to determine whether pre-fibrillar amyloid oligomers are present in drusen. To address this question, applicants used a recently developed “anti-oligomer” antibody that specifically recognizes pre-fibrillar oligomers, but not native or fibrillar protein. Although this antibody was initially raised against the Alzheimer Aβ peptide, it has been shown to also detect toxic oligomers from a variety of other amyloidogenic proteins, such as α-synuclein, islet amyloid polypeptide, prion peptide, polyglutamine, lysozyme, human insulin and, as recently demonstrated, the yeast prion protein. It has been suggested, therefore, that the pre-fibrillar amyloid oligomers from different proteins exhibit common structural features. Significantly, the antibody also exhibits a strongly protective effect against oligomer-induced toxicity, indicating that oligomers do indeed represent a toxic species.

The utility of this generic anti-amyloid oligomer antibody has been established in immunocytochemical studies as well. For example, through this antibody, the presence of pre-fibrillar oligomers has been demonstrated in AD-affected brains. These toxic oligomers were found to be in close proximity to senile plaques, yet have shown a distinct localization from the fibrillar plaque region, perhaps indicating the initial stage of amyloid fibril deposition. Furthermore, the same antibody was used in immunocytochemical studies to identify amyloid oligomers in the above-mentioned study on desmin-related cardiomyopathy.

In the present study, use of the anti-oligomer antibody has enabled us to detect the toxic oligomers within drusen-containing donor eyes, but not in control eyes without drusen. The presence of amyloid oligomers suggests that the underlying pathogenesis in AMD could be related to that of amyloid diseases.

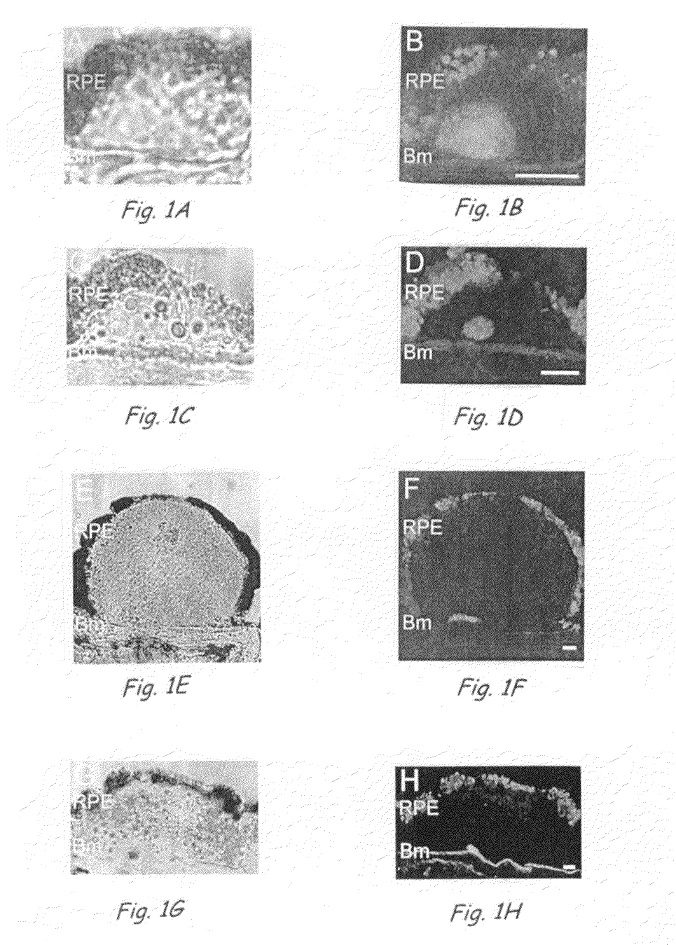

Immunofluorescent microscopy revealed the presence of amyloid oligomers in distinct areas of eyes that contained drusen. Antibody reactivity was most frequently observed centrally within drusen, wherein the fluorescent signal typically accumulated in close proximity to the inner collagenous layer of Bruch membrane and formed a distinct spherical subdomain. These structures, which we refer to as amyloid oligomer cores, did not vary significantly in size, even as the size of the drusen varied. In the smallest drusen (<20 μm), the amyloid oligomer cores predominantly occupied the drusen content (FIGS. 1A and B). In larger drusen, the cores remained at ˜15 μm in diameter, and retained the same spatial relationship abutting Bruch membrane whether they had the appearance of “hard” drusen (FIGS. 1 C-F), or macular soft drusen (FIGS. 1 G and H). These data suggest that the oligomer cores may occur at an early point during drusen biogenesis, but that they do not appear to grow as drusen become larger.

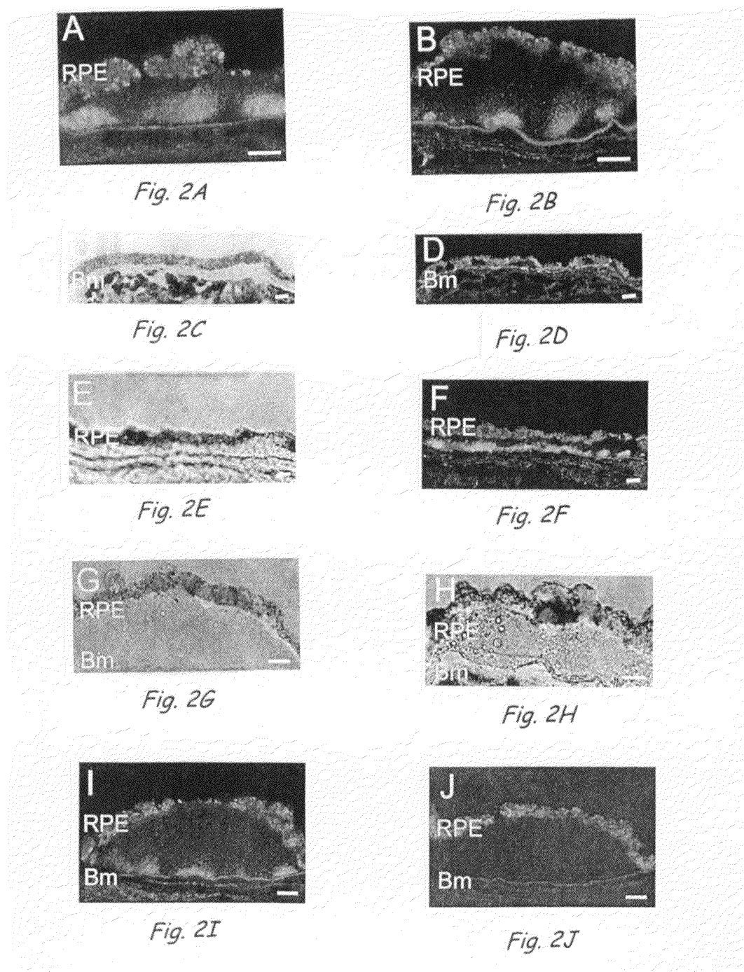

Although the size of the amyloid oligomer cores appeared to be restricted, the number of cores per druse did vary. Larger drusen, in particular, were sometimes observed to contain several amyloid oligomer cores (FIGS. 2 A, B and I), suggesting that these drusen may have formed from a coalescence of smaller drusen. Not all drusen, however, were observed to contain amyloid oligomer cores. However, given the small size of the amyloid oligomer cores relative to the larger drusen, many of the cores are likely to be out of the plane of section and thus not detectable. Table 1 summarizes the results obtained from eyes of 19 individuals. Strikingly, anti-oligomer antibody reactivity was observed in all eyes that contained drusen, but was not observed in eyes from age-matched controls or from those of young donors that did not contain drusen. These data establish a direct correlation between the presence of amyloid oligomers and drusen, and suggest a possible role for oligomer cores in drusen biogenesis.

Besides the presence of amyloid oligomer cores within drusen, oligomer staining was also observed at Bruch membrane in some cases, especially where it appeared to be thickened (FIGS. 2 C and D), and below Bruch membrane in basal linear deposits (FIGS. 2 E-H). Occasional staining within the RPE was also observed (FIG. 2 I). Staining was not observed in the neural retina (data not shown). Thus, antibody reactivity is also associated with additional pathological changes that are characteristic of AMD.

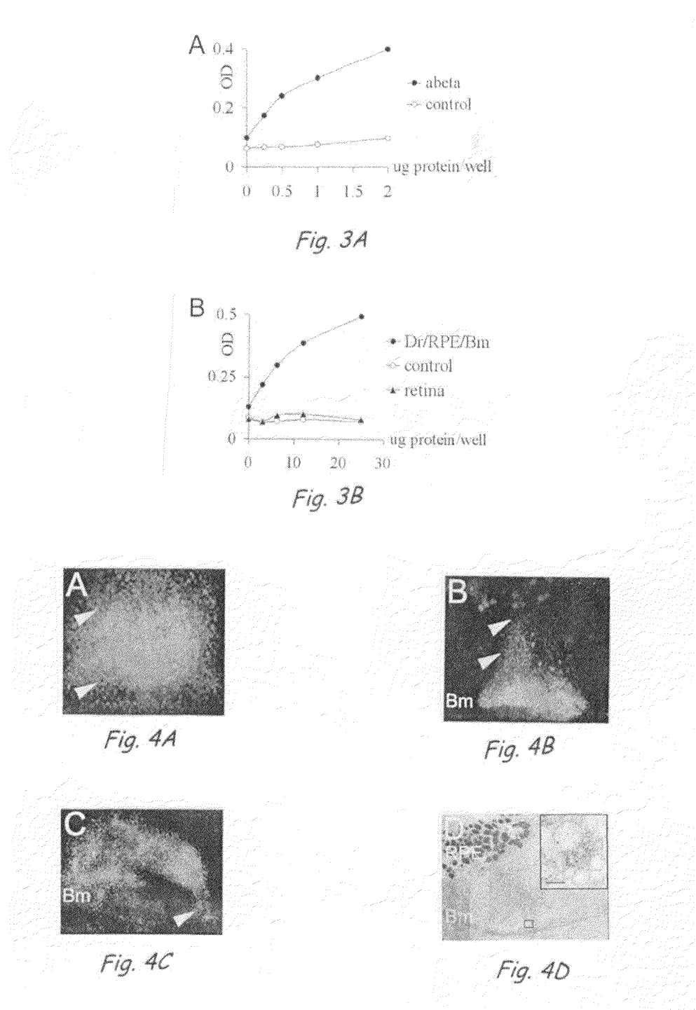

Specificity of the anti-oligomer antibody is exhibited in FIGS. 2 (I and J), which depicts serial sections obtained from the same druse. This druse contained several foci of anti-oligomer reactivity (FIG. 2 I). Staining was not seen when the section was incubated without the primary antibody (data not shown), nor when the antibody was pre-incubated with pre-fibrillar oligomers made from the Aβ₁₋₄₀ peptide (FIG. 2 J). We performed ELISA in order to further test the specificity of the anti-oligomer antibody in tissue homogenates prepared from the neural retina or from the underlying tissue containing drusen/RPE/Bruch membrane. In vitro synthesized pre-fibrillar Aβ₁₋₄₀ oligomers served as a positive control. As shown in FIG. 3A, a dose-dependent reactivity to increasing amounts of Aβ₁₋₄₀ oligomers was observed in the presence of the oligomer-specific antibody, but not when the antibody was omitted. Comparisons of antibody reactivity were also made between extracts prepared from the neural retina and from the tissue containing drusen/RPE/Bruch membrane (FIG. 3 B). A dose-dependent reactivity was observed with extracts prepared from drusen-containing tissue, whereas no reactivity was observed in the absence of the primary antibody. Little reactivity was observed with extracts prepared from the neural retina from the same donor eye. Thus, the positive signal seen in drusen appears to be highly specific for amyloid oligomers.

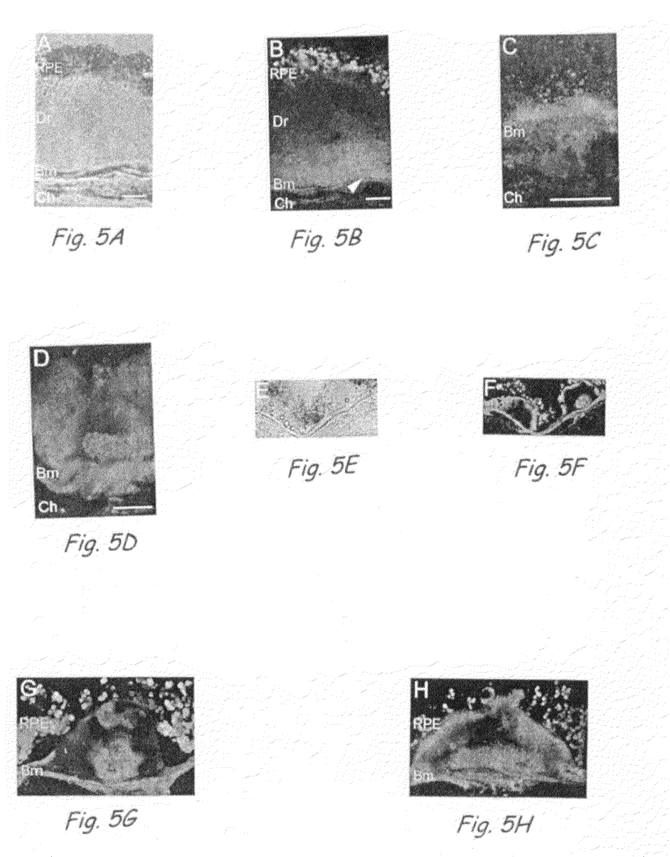

The data presented thus far support the notion that amyloid oligomers are present in drusen. Further inspections of the sections at higher magnifications, using laser scanning confocal microscopy, revealed a punctate pattern of small vesicular structures that increased in density toward the center of the amyloid oligomer core (FIG. 4 A). Occasionally, a decreasing gradation of the punctate pattern appeared to extend toward the RPE (FIG. 4 B). In other instances, the vesicular structures were observed to penetrate through the layers of Bruch membrane (FIG. 4 C). Together, these data suggest that the amyloid oligomers could be trafficked between the RPE cells, the drusen and the choroid.

In order to confirm the vesicular nature of the cores, we first used indirect immunofluorescence to identify a druse that contained an amyloid oligomer core. An adjacent serial section was then prepared for immunogold labeling, followed by electron microscopy (FIG. 4 D). As judged by distribution of the gold particles, these studies did indeed verify that the amyloid oligomers were associated with vesicular structures. Again, these vesicular structures appeared to be more concentrated near Bruch membrane, although similar structures were occasionally labeled within the apical aspect of the druse closer to the RPE as well (data not shown).

It has been reported in the literature that structures of the amyloid oligomer cores appear to be similar in some respects to dendritic cell processes. Double-staining with the anti-oligomer antibody (green, FITC) was performed in conjunction with anti-HLA-DR (red, Texas-red) in order to determine whether the immunoreactivities co-localized (FIG. 5 A-D). Strong HLA-DR reactivity can be seen within the drusen (FIG. 5 B), or beneath Bruch membrane, where it appears to penetrate into the druse and come into close proximity to the oligomer core (FIG. 5 C) and even completely surround the core (FIG. 5 D). In some instances, the HLA-DR reactivity appears to be in close proximity to the amyloid oligomer core (FIG. 5 B). Upon closer inspection, it is clear that the immunofluorescent signals do not overlap (FIG. 5 C and D). Thus, the amyloid oligomer cores are distinct structures from the HLA-DR positive dendritic cell processes described previously.

Double-staining was also performed on drusen sections to visualize oligomer cores and vitronectin, an acute phase protein that is a major component of drusen (38)(FIG. 5 E-H). All drusen stained positively for vitronectin, whereas oligomer cores were present only in a subset of drusen (e.g., FIG. 5 F). Vitronectin tends to have heterogeneous labeling patterns. In drusen that reacted positively for both oligomer cores and vitronectin, no overlap in their signals was observed. To ascertain whether the oligomer cores are assembled from amyloid Aβ(A), sections containing drusen were co-stained for these two components (FIG. 5 I-L). Most drusen contained either the Aβ assemblies or oligomer cores, but not both. Consistent with previous reports, Aβ reactivity was associated with vesicular structures within drusen (FIGS. 5 K and L). In one druse that reacted with both antibodies, the fluorescent signals did not co-localize: the amyloid oligomer reactivity was associated with the RPE, whereas the Aβ reactivity decorated spherical structures within the druse (FIG. 5 L). Thus, amyloid oligomers do not appear to co-localize with many of the known drusen components.

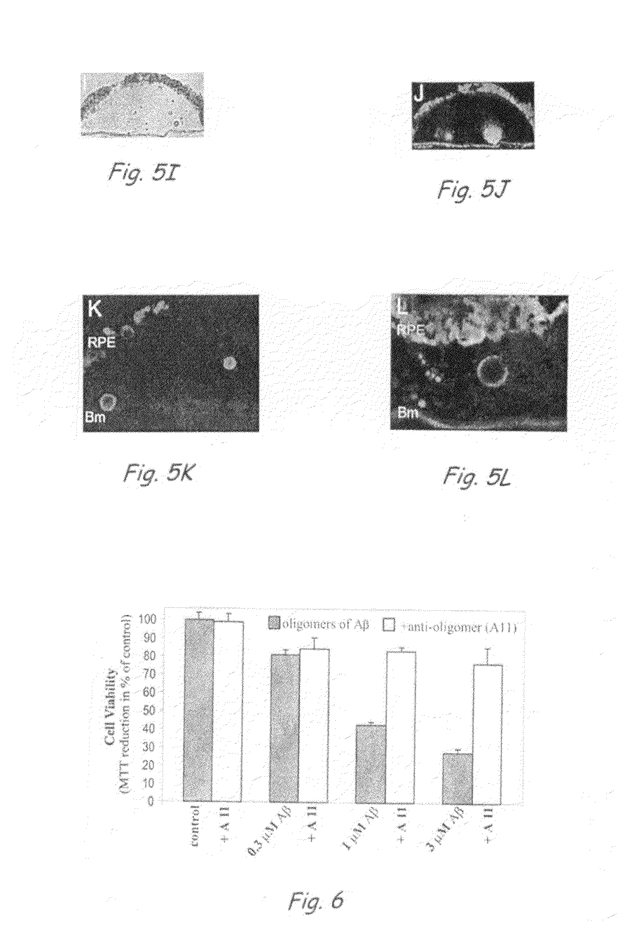

Different tissue or cultured cell types show varying susceptibility to the toxicity of amyloid aggregates. We sought to examine whether amyloid oligomers are toxic to RPE cells, given their close proximity to each other in eyes that contain drusen. FIG. 6 shows that soluble oligomers made from A 140 are indeed toxic to human primary RPE cells in culture. This toxicity is largely blocked if the Aβ anti-oligomer antibody is included in the incubation mixture. Thus, the presence of amyloid oligomers in close proximity to RPE cells may have a negative impact on the physiology of these cells during drusen biogenesis and in AMD.

Pre-fibrillar oligomeric structures made from amyloidogenic proteins or peptides are thought to contribute to the pathogenesis of amyloid diseases. Such structures can be detected in tissue sections in situ by a recently developed conformation-specific, but not sequence-specific, antibody (24). Through the use of this antibody, we demonstrated the presence of amyloid oligomers in drusen-containing eyes and eyes that have been clinically diagnosed with atrophic AMD (Table 1). Importantly, no reactivity was observed in control eyes without drusen, which suggests that the formation of amyloid oligomers is a disease-specific process. Since pre-fibrillar amyloid oligomers demonstrate toxicity toward cultured primary human RPE cells, they may contribute to their demise during the disease process. Thus, AMD and amyloid diseases appear to share similar protein misfolding events, and may share common pathogenic pathways as well.

One commonality is the discovery that spherical Aβ assemblies, as well as other pro-inflammatory proteins commonly seen in AD plaques, are also present in drusen. In particular, it has been reported that a single druse may contain no Aβ structures or a large number of them, ranging in diameter from 0.25 to 10 μm and displaying highly organized concentric layers when viewed under an electron microscope. Thus, the literature has described Aβ assemblies that are structurally distinct from the oligomer-associated vesicles due to differences in their size, shape and distribution. Indeed, our data show that they do not co-localize in drusen. It is important to note, however, that the epitope for Aβ may have been masked within the oligomeric structure, as is the case when Aβ monomers are transformed into amyloid fibrils. Therefore, we cannot preclude the possibility that the oligomeric cores in drusen consist of Aα. Another drusen subdomain is comprised of core-like structures that exhibit Arachea hypogea agglutinin (PNA) reactivity. Although these structures to some extent resemble the oligomeric structures described herein, they ranged in diameter from 5 to 38 μm, whereas the amyloid oligomer cores are typically 10 to 15 μm. It appears, then, that the oligomeric structures discussed here differ distinctively from the substructures within drusen that had been described previously. Composition of the oligomeric structures within drusen has yet to be determined and is under investigation.

Applicants' data have yielded further evidence that AMD and amyloid diseases share common pathogenic pathways, although amyloid fibrils have not been observed in drusen. Amyloid protein-related toxicity in the absence of fibrils had been observed in the past. For example, in a transgenic mouse model for AD, overexpression of the human wild-type β-Amyloid Precursor Protein leads to learning deficits and Aβ deposition without amyloid plaque formation. In the case of a mouse model for PD, it has been shown that overexpression of wild-type α-synuclein results in motor abnormalities and the formation of α-synuclein-containing, non-fibrillar inclusions. In transgenic rats expressing human islet amyloid polypeptide that served as an animal model for type 2 diabetes, apoptosis of pancreatic islet cells did not correlate with amyloid formation. These results indicate that the presence of amyloid fibrils is not a prerequisite for pathogenesis and implicate the toxic pre-fibrillar oligomers as an underlying cause of cell loss. Thus, AMD and desmin-related cardiomyopathy might be just two of perhaps several diseases that are related to amyloid diseases, yet do not exhibit noticeable amyloid fibril deposition.

Although it is not obvious why amyloid fibrils are difficult to detect in drusen, the rates at which oligomers and fibrils are turned over are likely to be of importance. As mentioned above, amyloid fibril formation is a stepwise process, and the overall yield of oligomers and fibrils depends upon the underlying kinetics of each step. Thus, two possible explanations for the low degree of fibril deposition are slow rates of fibril formation or fast rates of clearance. It is known that rates of amyloid fibril formation are largely dictated by experimental conditions and biochemical data suggest that, under appropriate conditions, the stability of oligomers can be maintained for extended periods of time before they convert into fibrils. It is also conceivable that oligomers might be cleared out of drusen before they can be converted into fibrils. Although the present study provides no direct evidence of such clearance, the ability of oligomeric structures to penetrate through Bruch membrane suggests this possibility.

Recent immunocytochemical data on HLA-DR reactivity in drusen suggest the presence of dendritic cell processes in drusen. Dendritic cells are antigen-presenting cells that take up foreign substances and, in principle, may facilitate the clearance of amyloid oligomers. Indeed, our studies show the presence of HLA-DR reactive structures in drusen similar to that reported by Hageman et al. These putative dendritic cell processes appear more frequently in and around the drusen than the amyloid oligomers, and they were sometimes found in close proximity to the amyloid oligomer cores. They do not, however, appear to co-localize.

Applicants' data add to the growing list of evidence that reveals similarities between AMD and amyloid diseases. It is particularly noteworthy that pro-inflammatory proteins have been identified in the extracellular deposits associated with these diseases. Evidence of complement activation has been observed within certain RPE cells, small drusen and large soft drusen that are present in aging eyes as well as in AMD eyes. This observation has led to the hypothesis that aberrant immune reactions may play a role in drusen biogenesis. Significantly, a polymorphism in complement factor H, a key regulator of complement activation, has recently been identified as a major risk factor for AMD. This finding has placed a significant focus on the role of complement activation in the pathogenesis of AMD. What may be the factors that lead to the activation of the immune response? Here, we report the presence of amyloid oligomers in a similar distribution of drusen, RPE cells and basal deposits. It is noteworthy that these oligomers have been implicated in the pathogenesis of amyloid diseases due to their demonstrated toxicity toward cells. It is possible that the presence of oligomers in close proximity to RPE cells may compromise their function, leading to activation of the complement cascade and formation of drusen.

In summary, the presence of amyloid oligomers in drusen suggests that AMD and amyloid diseases share commonalities with respect to protein misfolding and pathogenesis. AMD and desmin-related cardiomyopathy may well come to represent the first examples of a new class of amyloid disease in which oligomeric intermediates, rather than mature amyloid fibrils, accumulate.

Human tissue. Intact human donor eyes were obtained from the Oregon Lions Sight & Hearing Foundation (Portland) and the Alzheimer Disease Research Center Neuropathology Core (University of Southern California). Eyes from 19 individuals were examined, four of which had documented clinical histories of AMD as shown in Table 1 below:

| A | 87 | M | no data | + | + |
|---|---|---|---|---|---|
| | 86 | M | unremarkable | + | + |
| | 88 | F | macular | + | + |
| | | | degeneration | | |
| | 96 | F | no data | + | + |
| | 87 | F | no data | + | + |
| | 94 | F | no data | + | + |
| | 92 | M | unremarkable | + | + |
| | 92 | F | pseudophakia | + | + |
| | 75 | F | Macular | + | + |
| | | | degeneration | | |
| | 83 | M | IOL surgery | + | + |
| | 82 | F | macular | + | + |
| | | | degeneration | | |
| | 98 | M | macular | + | + |
| | | | degeneration | | |
| | 82 | F | cataract | + | + |
| | 77 | M | unremarkable | + | + |
| | 87 | M | unremarkable | − | − |
| | Age | Sex | Ocular history | drusen | oligomers |
| B | 81 | F | cataract | − | − |
| | 56 | F | unremarkable | − | − |
| | 21 | F | unremarkable | − | − |
| | fetus | | | − | − |

The data summarized in Table 1 shows that oligomer reactivity is specific for drusen-containing tissue, Whole eyes from 19 donors were screened by confocal microscopy for the presence of amyloid oligomers. Oligomer reactivity is observed only when drusen are present. No reactivity is observed in age-matched control eyes without drusen, or in eyes from young donors that do not contain drusen.

All eyes were kept at 4° C. and processed at less than 24 hours postmortem. Fixation was avoided since it would have interfered with antigen detection using the anti-oligomer-specific antibody. After removing the anterior pole, the retina was peeled off and the posterior pole of the eyeball was examined under a dissecting microscope (MZ125, Leica, Germany) for the presence of drusen. All areas containing drusen were included. Tissue was cut into 1 cm×0.5 cm rectangles, using a coated stainless steel razor blade, and embedded in O.C.T. (Tissue-Tek, Sakura Finetich, Torrance, Calif.).

Confocal immunofluorescence microscopy. Frozen embedded tissue was sectioned on a cryostat (Leica CM 3050S, Germany) at −20° C. Frozen sections, 8-10 μm thick, were collected on precleaned superfrost^{R} plus-slides (VWR Scientific, West Chester, Pa.), air-dried for 30 minutes, and stored at −20° C. Immunocytochemical studies using the anti-oligomer-specific antibody were performed as described previously. Briefly, sections were blocked overnight at 4° C. in blocking solution (phosphate-buffered saline containing 2% BSA and 2% goat serum), and incubated the following day with affinity-purified anti-oligomer-specific antibody (1.6 mg/ml) for one hour at room temperature. Sections were then washed and incubated with a fluorescein-conjugated goat anti-rabbit antibody (1:100, Vector Laboratories, Burlingame, Calif.) for one hour at room temperature. In order to detect oligomers and HLA-DR or drusen components such as vitronectin and Aβ, sections were processed as mentioned above and incubated with mouse anti-human HLA-DR antibody (0.5 mg/ml, Pharmingen, San Diego, Calif.), mouse anti-vitronectin:antibody (1:200, Biosource) or with mouse anti-Aβ, antibody (1:100, 4G8, Signet Laboratories), which is directed against the residues 17-24 of the Aβ, peptide. Digital images of immunostained sections were acquired on an LSM 510 Zeiss laser scanning confocal microscope (Thornwood, N.Y.).

Electron microscopy. Pre-fibrillar oligomers were first identified in frozen sections using immunofluorescence. Adjacent serial sections known to contain oligomers were incubated with the anti-oligomer antibody, and subsequently with 5 nm gold-conjugated goat anti-rabbit antibody (Ted Pella, Redding, Calif.). The sections were washed and pre-embedded in 4% agarose. Agarose-embedded sections were then briefly fixed in OsO₄, dehydrated in increasing concentrations of ethanol, infiltrated with epoxy resin, and sectioned at 70 nm using an ultramicrotome (Ultracut UCT; Leica, Germany) for electron microscopy. Images were obtained using a transmission electron microscope (EM10; Zeiss, Germany).

Preparation of Soluble Aβ oligomers. Aβ oligomers were prepared in accordance with techniques known in the art. Briefly, 1.0 mg Aβ was dissolved in 400 μl 1,1,1,3,3,3-hexafluoro-2-propanol (HFIP) for 10 minutes at room temperature. Aliquots (100 μl) of the solution were added to 900 μl ddH₂O in siliconized Eppendorf tubes. After 10 minutes of incubation, the samples were centrifuged for 15 minutes at 14,000 g and the supernatant fraction was transferred to a new siliconized tube. The HFIP was evaporated by blowing under an N₂ stream for 5 to 10 minutes. The samples were then stirred at 500 rpm using a Teflon coated micro stir bar for 24-48 hr at room temperature. Aliquots were taken at 6-12 hr intervals to check for the presence of spherical oligomers.

Enzyme-Linked Immunosorbent Assay (ELISA). ELISA was performed with tissue homogenates and oligomers made from the Aβ peptide. To process eye tissue samples, neural retina was peeled off the underlying RPE/BM/choroid-complex at the posterior eye pole. Isolated tissues (neural retina or the underlying RPE/BM/choroid-complex) were homogenized using a tip sonicator (Microson™) in ddH₂O, centrifuged, and supernatant was collected. ELISA was performed using the anti-oligomer-specific antibody, as described by Kayed and colleagues. Briefly, samples were diluted in coating buffer (0.1 M sodium bicarbonate) and added to wells of a 96-well microplate (Becton Dickinson, Franklin Lakes, N.J.). After two hours of incubation at 37° C., samples were blocked for two hours at 37° C. with 3% BSA TBS-T. One hundred μl of anti-oligomer antibody (1:2500) were added and incubated at 37° C. for one hour, prior to incubation with 100 μl of horseradish peroxidase-conjugated anti-rabbit IgG for one hour at 37° C. Subsequent to development with 3,3′,5,5′-tetramethylbenzidine (TMB), the reaction was stopped with 100 μl 1M HCl, and plates were read at 450 nm (Benchmark Plus, Bio-Rad Laboratories, Hercules, Calif.).

Cell viability assay. Cell viability was assessed spectrophotometrically using an MTT (3-[4,5-dimethylthiazol-2-yl]-2,5-diphenyl tetrazolium bromide)-based assay (Sigma-Aldrich, St. Louis, Mo.). RPE cells isolated from human fetal eyes were obtained from Advanced Bioscience Resources, Inc. (Alameda, Calif.). The cells were maintained in Dulbecco's modified Eagle's medium (DMEM) supplemented with 2 mM L-glutamine, 100 U/ml penicillin, 100 μg/ml streptomycin, and 10% fetal bovine serum at 37° C. Third to fourth passage cells were seeded at 10,000 cells per well in a 96-well plate and grown for 3 to 4 days to ˜90% confluence. Prior to the toxicity assay, media was replaced with indicated concentrations of Aβ oligomers alone or with equal molars of the Aβ anti-oligomer antibody dissolved in phenol red-free DMEM. The conditions were carried out in triplicate. After four hours, MTT dissolved in DMEM as added to the cells and incubated for an additional four hours. Insoluble crystals were dissolved by adding MTT solubilization solution (10% Triton X-100, 0.1 N HCl in anhydrous isopropanol) and absorbance was measured at 570 nm.

### Antibody Preparation and Therapeutic Applications

Each of the following amyloid peptides have been shown to form amyloid peptide aggregates which produce a conformational epitope recognized by the antibodies of the present invention, for example, antibodies produced against [symbol] peptide oligomeric intermediates. Some of these peptides are present in amyloid deposits of humans or animals having a disease characterized by the amyloid deposits. The present invention is not limited to the listed peptide or protein sequences or the specific diseases associated with some of the sequences. The present invention contemplates antibodies as described herein binding to other amyloid peptide aggregates or all other amyloid peptide aggregates. In particular, the present invention contemplates and includes the application of methods and compositions of the present invention to other peptide or protein sequences which form amyloid precursor aggregates associated with other diseases.

A40

(SEQ ID NO 1)

DAEFRHDSGYEVHHQKLVFF AEDVGSNKGA IIGLMVGGVV
A42

(SEQ ID NO 2)

| | |
|---|---|
| DAEFRHDSGY EVHHQKLVFF AEDVGSNKGA IIGLMVGGVV IA | > |

Human IAPP

(SEQ ID NO 3)

KCNTATCATQ RLANFLVHSS NNFGAILSST NVGSNTY
Human Prion 106-126

(SEQ ID NO 4)

KTNMKHMAGA AAAGAVVGGL G

Stefani and coworkers (Bucciantini et al (2002) Nature 416, 507-511) have recently reported that amyloid peptide aggregates formed from non-disease-related proteins are inherently cytoxic, suggesting that they may have a structure in common with disease related amyloid peptides. Non-disease related amyloid peptide aggregates comprising the following non-disease related amyloid peptides are also shown to bind to the antibodies of the present invention.

Poly glutamine synthetic peptide KK(Q40)KK

(SEQ ID NO 5)

KKQQQQQQQQ QQQQQQQQQQ QQQQQQQQQQ QQQQQQQQQQ
QQKK
Human Lysozyme

(SEQ ID NO 6)

MKALIVLGLV LLSVTVQGKV FERCELARTL KRLGMDGYRG
SLANWMCLA KWESGYNTRA TNYNAGDRST DYGIFQINSR
YWCNDGKTPG AVNACHLSCS ALLQDNIADA VACAKRVVRD
PQGIRAWVAW RNRCQNRDVR QYVQGCGV
Human Insulin

(SEQ ID NO 7)

MALWMRLLPL LALLALWGPD PAAAFVNQHL CGSHLVEALY
LVCGERGFFY TPKTRREAED LQVGQVELGG GPGAGSLQPL
ALEGSLQKRG IVEQGCTSIC SLYQLENYCN
Human Transthyretin

(SEQ ID NO 8)

MASHRLLLLC LAGLVFVSEA GPTGTGESKC PLMVKVLDAV
RGSPAINVAV HVFRKAADDT WEPFASGKTS ESGELHGLTT
EEEFVEGIYK VEIDTKSYWK ALGISPFHEH AEVVFTANDS
GPRRYTIAAL LSPYSYSTTA VVTNPKE
Human Alpha Synuclein

(SEQ ID NO 9)

MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

In addition, oligomeric intermediates formed from variants and fragments of wild type [symbol] 42, [symbol] 40 including, without limitation [symbol] 42 (A21G) Flemish mutation), [symbol] 42 (E22Q) Dutch mutation, [symbol] 42 (E22G) Arctic mutation, [symbol] 42 (D23N) Iowa mutation, [symbol] 40 (A21G) Flemish mutation), [symbol] 40 (E22Q) Dutch mutation, [symbol] 40 (E22G) Arctic mutation, [symbol] 40 (D23N) Iowa mutation, [symbol] 40 (E22Q &D23N) Dutch & Iowa mutations, [symbol] 3-42 (pGlu 3), [symbol] 3-40 (pGlu 3), [symbol] 8-42, [symbol] 17-42, [symbol] 1-16, [symbol] 3-11, [symbol] 25-35, [symbol] 4-16 (3 analogues, CyS¹⁶ [symbol] 4-16, Ala⁴ [symbol] 4-16, and Ala¹⁰ [symbol] 4-16), His6 [symbol] 40C40 (6 histidines appended to the amino terminus of AβC40) are recognized by the antibodies of the present invention. Other oligomeric intermediates recognized by antibodies of the invention include, without limitation, oligomeric intermediates formed from IAPP(C2A and C7A) where alanine is substituted for the naturally occurring cysteine in IAPP, Polyglutamine KKQ40KK or poly glutamine where the number of Q residues is greater than 32, Calcitonin, TTR and its mutants TTR Pro⁵⁵, TTR Phe⁷⁸, vitronictin, poly Lysine, poly arginine, serum amyloid A, cystantin C, IgG kappa light chain, oligomeric intermediates produced from other amyloid peptides disclosed herein and amyloid intermediates associated with amyloid diseases disclosed herein.

The present invention provides for amyloid disease therapeutics which induce a specific immune response against amyloid oligomeric intermediates. Therapeutics of the invention include antibodies that specifically bind to oligomeric intermediates. Such antibodies can be monoclonal as described in this application or polyclonal as described in PCT International Application No. PCT/US2003/028829, which is incorporated herein by reference. In one useful embodiment, the antibodies bind to a conformational epitope. The production of non-human monoclonal antibodies of the present invention (e.g., murine or rat) can be accomplished by, for example, immunizing the animal with an oligomeric intermediate mimic of the invention. Also contemplated is immunizing the animal with a purified amyloid intermediate.

Humanized forms of mouse antibodies of the invention can be generated by linking the CDR regions of non-human antibodies to human constant regions by recombinant DNA techniques. See Queen et al., Proc. Natl. Acad. Sci. USA 86, 10029-10033 (1989) and WO 90/07861 (incorporated by reference for all purposes).

Human antibodies may be obtained using phage-display methods. See, for example, Dower et al., WO 91/17271 and McCafferty et al., WO 92/01047. In these methods, libraries of phage are produced in which members display different antibodies on their outer surfaces. Phage displaying antibodies with a desired specificity are selected by affinity enrichment. Human antibodies against oligomeric intermediates may also be produced from non-human transgenic mammals having transgenes encoding at least a segment of the human immunoglobulin locus and an inactivated endogenous immunoglobulin locus. See, for example, Lonberg et al., WO93/12227 (1993); Kucherlapati, WO 91/10741 (1991) (each of which is incorporated by reference in its entirety for all purposes). Human antibodies can be selected by competitive binding experiments, or otherwise, to have the same epitope specificity as a particular mouse antibody. Such antibodies are particularly likely to share the useful functional properties of the mouse antibodies.

Human or humanized antibodies can be designed to have IgG, IgD, IgA and IgE constant region, and any isotype, including IgG, IgG2, IgG3 and IgG4. Antibodies can be expressed as tetramers containing two light and two heavy chains, as separate heavy chains, light chains, as Fab, Fab′ F(ab′)₂ and Fv, or as single chain antibodies in which heavy and light chain variable domains are linked through a spacer.

In certain instances it may be desirable to combine one or more amyloid peptide aggregate immunogens of the present invention with a suitable carrier. Suitable carriers include serum albumins, keyhole limpet hemocyanin, immunoglobulin molecules, thyroglobulin, ovalbumin, tetanus toxoid, or a toxoid from other pathogenic bacteria, such as diphtheria, E. coli, cholera, or H. pylori, or an attenuated toxin derivative. Other carriers which may act as adjuvants for stimulating or enhancing an immune response include cytokines such as IL-1, IL-1 and peptides, IL-2, INF, IL-10, GM-CSF, and chemokines, such as M1P1 and RANTES.

Human or animal subjects or patients amenable to treatment with monoclonal antibodies of the present invention include individuals at risk of amyloid disease but not showing symptoms, as well as those who already show symptoms or other evidence of amyloid disease. In the case of certain amyloid diseases including AD, virtually anyone is at risk of suffering from the disease.

Therefore, monoclonal antibodies as described herein, or similar polyclonal antibodies as described in parent application Ser. No. 10/527,678 (PCT International Publication WO2004/024090) or immunogens capable of eliciting an antibody response to the conformation epitope of amyloid oligomers in a subject could be administered prophylactically, to the general population without any assessment of the risk of the subject patient. The present methods are especially useful for individuals who do have a known genetic risk of a disease that is associated with or is known to have co-morbitiy with drusen or drusen-like deposits. Such individuals may have been diagnosed with or may have risk factors (e.g., family history, genetic markers, etc.) for the development of AMD, membranoproliferative glomerulonephritis type II, elastosis, other amyloid diseases, etc. For example, genetic markers of risk toward AD include mutations in the APP gene, particularly mutations at position 717 and positions 670 and 671 referred to as the Hardy and Swedish mutations respectively (see Hardy, TINS, supra). Other markers of risk for AD are mutations in the presenilin genes, PS1 and PS2, and ApoE4, family history of AD, hypercholesterolemia or atherosclerosis.

Symptoms of AMD, membranoproliferative glomerulonephritis type II, elastosis, AD and other amyloid diseases are apparent to many physicians. For example, individuals presently suffering from AMD are often diagnosed during routine eye examinations. In addition, a number of diagnostic tests are available for identifying individuals who have amyloid diseases. For example, in the case of AD these include measurement of CSF tau and A42 levels. Elevated tau and decreased [symbol] 42 levels signify the presence of AD.

In prophylactic therapy applications, the compositions of this invention or medians are administered to patients who are believed to be susceptible to, or who have risk factors for AMD, membranoproliferative glomerulonephritis type II, elastosis, AD and other amyloid diseases, in amounts sufficient to eliminate or reduce the risk or delay the outset of the disease. In other therapeutic applications, the compositions or medians of this invention are administered to patients in whom drusen or drusen-like deposits have been observed or who already exhibit signs or symptoms of AMD, membranoproliferative glomerulonephritis type II, elastosis, AD or other diseases or disorders that are associated with drusen or drusen-like deposits, in amounts sufficient to inhibit the formation or biosynthesis of the drusen or drusen-like deposits and/or cause regression of existing drusen or drusen-like deposits and/or cure or lessen the severity of AMD, membranoproliferative glomerulonephritis type II, elastosis, AD or other diseases or disorders that are associated with drusen or drusen-like deposits. An amount adequate to accomplish this is defined as a therapeutically or pharmaceutically effective dose. In both prophylactic and therapeutic regimes, the treatments of the present invention may be administered in repeated dosages until a sufficient immune status has been achieved. Typically, the patient's immune status will be monitored and further dosages will be given if the immune status starts to fade.

Effective doses of the compositions of the present invention, for the treatment of the above described conditions vary depending upon many different factors, including means of administration, target site, physiological state of the patient, whether the patient is human or animal, other medications administered, and whether treatment is prophylactic or therapeutic. Usually, the patient is a human, but in some diseases, such as mad cow disease, the patient can be a nonhuman mammal, such as a bovine or in the case of Alzheimer's disease, the patient may be a dog. Treatment dosages need to be titrated to optimize safety and efficacy. For passive immunization with an antibody, the dosage ranges from about 0.0001 mg/kg of body weight to about 100 mg/kg of body weight, and more usually about 0.01 mg/kg of body weight to about 5 mg/kg of body weight of the host. The amount of immunogen to be administered may depend on whether any adjuvant is also administered, with higher dosages being required in the absence of adjuvant. For example, 0.1 to 100 cc of a solution containing approximately 1% by weight of the desired immunogen my be injected subcutaneously, thereby delivering a dose of 1 mg to 1 g of the immunogen per injection. The timing of injections can vary significantly from once a day, to once a year, to once a decade. One typical regimen for administration of immunogen consists of an immunization followed by booster injections at 6 weekly intervals. Another regimen consists of an immunization followed by booster injections 1, 2 and 12 months later. Another regimen entails an injection every two months for life. Alternatively, booster injections can be on an irregular basis as indicated by monitoring of immune response.

Therapeutics for inducing an immune response can be administered by any suitable route of administration, for example, parenteral, topical, intravenous, oral, subcutaneous, intraperitoneal, intranasal or intramuscular. The most typical route of administration is subcutaneous although others can be equally effective. The next most common is intramuscular injection. This type of injection is most typically performed in the arm or leg muscles. Intravenous injections as well as intraperitoneal injections, intraarterial, intracranial, or intradermal injections may also be effective in generating an immune response. In some methods, therapeutics are injected directly into a particular tissue where deposits have accumulated or may accumulate.

Monoclonal antibodies of the invention can optionally be administered in combination with other agents that are at least partly effective in treatment of amyloidogenic disease. In the case of Alzheimer's and Down's syndrome, in which amyloid deposits occur in the brain, therapeutics of the invention can also be administered in conjunction with other agents that increase passage of the compositions of the invention across the blood-brain barrier. For example, as described in detail herebelow, anti-inflammatory dosages of colloidal gold or gold salts may be administered concomitantly (e.g., before, concurrently with or after) the monoclonal antibody to deter the brain inflammation associated with AD and other amyloid diseases.

Immunogens of the invention may sometimes be administered in combination with an adjuvant. A variety of adjuvants can be used in combination with an immunogen of the invention to elicit an immune response. Preferred adjuvants augment the intrinsic response to an immunogen without causing conformational changes in the immunogen that affect the qualitative form of the response. Preferred adjuvants include alum, 3 de-O-acylated monophosphoryl lipid A (MPL) (see GB 2220211). QS21 is a triterpene glycoside or saponin isolated from the bark of the Quillaja Saponaria Molina tree found in South America (see Kensil et al., in Vaccine Design: The subunit and Ajuvant Approach (eds. Powell & Newman, Plenum Press, NY, 1995); and U.S. Pat. No. 5,057,540). Other adjuvants are oil in water emulsions, such as squalene or peanut oil, optionally in combination with immune stimulants, such as monophosphoryl lipid A. See, for example, Stoute et al., N. Engl. J. Med. (1997) 336, 86-91. Another useful adjuvant is CpG described in Bioworld Today, Nov. 15, 1998. Alternatively, a immunogen can be coupled to an adjuvant. However, such coupling should not substantially change immunogen so as to affect the nature of the immune response thereto. Adjuvants can be administered as a component of a therapeutic composition with an active agent or can be administered separately, before, concurrently with, or after administration of the therapeutic.

A preferred class of adjuvants is aluminum salts (alum), such as aluminum hydroxide, aluminum phosphate, aluminum sulfate. Such adjuvants can be used with or without other specific immunostimulating agents such as MPL or 3-DMP, QS21, polymeric or monomeric amino acids such as polyglutamic acid or polylysine.

Another class of adjuvants is oil-in-water emulsion formulations. Such adjuvants can be used with or without other specific immunostimulating agents such as muramyl peptides (for example, N-acetylmuramyl-L-threonyl-D-isoglutamine (thr-MDP), -acetyl-normuramyl-L-alanyl-D-isoglutamine (nor-MDP), N-acetylmuramyl-L-alanyl-D-isoglutamyl-L-alanine-2-(1′-2′dipalmitoyl-sn-glycero-3-hydroxyphosphoryloxy)-ethylamine (MTP-PE), N-acetylglucsaminyl-N-acetylmuramyl-L-AI-D-isoglu-L-Ala-dipalmitoxy propylamide (DTP-DPP) theramide™), or other bacterial cell wall components. Oil-in-water emulsions include (a) MF59 (WO 90/14837), containing 5% Squalene, 0.5% Tween 80 and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalane, 0.4% Tween 80.5% pluroinic-blocked polymer L121, and thr-MDP, either microfluidized into a submicron emulsion or vortexed to generate a larger particle size emulsion, and (c) Ribi™ adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™)

Another class of preferred adjuvants is saponin adjuvants, such as Stimulons (QS21, Aquila, Worcester, Mass.) or particles generated therefrom such as ISCOMs (immunostimulating complexes) and ISCOMATRIX. Other adjuvants include Complete Freund's Adjuvant (CFA) and Incomplete Freund's Adjuvant (IFA). Other adjuvants include cytokines, such as interleukins, for example, IL-1, IL-2, and IL-12, macrophage colony stimulating factor (M-CSF), tumor necrosis factor (TNF) and/or chemokines such as CXCL10 and CCL5.

An adjuvant can be administered with an immunogen as a single composition, or can be administered before, concurrent with or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the vaccine containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Optionally, two or more different adjuvants can be used simultaneously. Preferred combinations include alum with MPL, alum with QS21, MPL with QS21, and alum, QS21 and MPL together. Also, Incomplete Freund's adjuvant can be used (Chang et al., Advanced Drug Delivery Reviews 32, 173-186 (1998)), optionally in combination with any of alum, QS21, and MPL and all combinations thereof.

Compositions of the invention are often administered as pharmaceutical compositions comprising a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonmonoclonal antibodyic stabilizers and the like. However, some reagents suitable for administration to animals, such as complete Freund's adjuvant are not typically included in compositions for human use.

Pharmaceutical compositions can also include large, slowly metabolized macromolecules such as proteins, polysaccharides, polylactic acids, polyglycolic acids and copolymers (such as latex functionalized sepharose, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent or pharmaceutical carrier which can be a sterile liquid such as water oils, saline, glycerol, or ethanol.

Auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Compositions may be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. See Langer, Science (1990) 249, 1527 and Hanes, Advanced Drug Delivery Reviews (1997) 28, 97-119. The compositions of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to about 10%, for example, about 1% to about 2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and may contain about 10% about 95% of active ingredient, for example, about 25% to about 70%.

Topical application can result in transdermal or intradermal delivery. Topical administration can be facilitated by co-administration of the composition with cholera toxin or detoxified derivatives or subunits thereof or other similar bacterial toxins. See Glenn et al., Nature (1998) 391,851. Co-administration can be achieved by using the components as a mixture or as linked molecules obtained by chemical crosslinking or expression as a fusion protein.

Alternatively, transdermal delivery can be achieved using a skin path or using transferosomes. See for example, Paul et al., Eur. J. Immunol. (1995) 25, 3521-24; Cevc et al., Biochem. Biophys. Acta (1998) 1368, 201-15.

### Concomitant Administration of Gold or Other Antiinflammatory

The anti-inflammatory effects of gold are well established. For example, injectable colloidal gold preparations (Myochrysine™ or Solganal™) are commercially available for the treatment of rheumatoid arthritis. A gold preparation for oral administration (Auranofin™) is also available. Inflammation of in the brain is thought to be a cause or contributing factor Alzheimer's Disease, primarily because the Aβ which is found in the brains of Alzheimer's patients is known to be an inflammatory protein. In view of this, others have proposed the use of non-steroidal anti-inflammatory drugs such as rofecoxib (Vioxx) and naproxen (Aleve) to slow the progression of Alzheimer's Disease. Similarly, inflammation is believed to play a role in at least some chorioretinal disorders, such as AMD, and some studies have indicated that patients who routinely take anti-inflammatory drugs (e.g., non-steroidal anti-inflammatory agents, statins) have a lower incidence of AMD.

Applicants have determined, on the basis of histopathological observations, that the subcutaneous administration of colloidal gold can reduce microglial activation in the brains of mice modeling for amyloid disease. The present invention includes the administration of colloidal gold, gold salts or other antiinflammatory agents to the subject in an amount that is therapeutically effective to decrease neural inflammation. In some cases, the gold or anti-inflammatory agent may be combined with the monoclonal antibody or immunogen. In other cases, the gold or anti-inflammatory agent may be administered separately from the monoclonal antibody or immunogen. Any suitable dose, dosing schedule or route of administration may be used. For example, commercially available gold preparations for treatment of rheumatoid arthritis may be administered by the same routes of administration (subcutaneous injection of Myochrysine™ or Solganal™ or oral administration of Auranofin™ and dosages/dosing schedules recommended for treatment of rheumatoid arthritis.

Although the foregoing invention has been described in detail for purposes of clarity of understanding, it will be obvious that certain modifications may be practised within the scope of the appended claims. All publications and patent documents cited herein are hereby incorporated by reference in their entirety for all purposes to the same extent as if each were so individually denoted.

What is claimed is:

1. A method for inhibiting the formation and/or biosynthesis of, or for causing diminution of, drusen or a drusen-like deposit in a human or animal subject or for preventing or treating a disease or disorder that is associated with drusen or drusen-like deposits, said method comprising the step of:
(A) administering to the subject, in a therapeutically effective amount, a composition that comprises at least one of:
i) a conformational epitope of an aggregate that contributes to the formation or biosynthesis of drusen or drusen-like deposits; and
ii) an antibody that binds to a conformational epitope of an aggregate that contributes to the formation or biosynthesis of drusen or the drusen-like deposit.

2. A method according to claim 1 wherein Step A comprises inducing an immune response against the conformational epitope.

3. A method according to claim 1 wherein the composition administered in Step A comprises a peptide.

4. A method according to claim 3 wherein the peptide is conformationally constrained.

5. A method according to claim 3 wherein the peptide comprises an amino acid sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and combinations thereof.

6. A method according to claim 3 wherein the peptide comprises SEQ ID NO. 1.

7. A method according to claim 1 wherein the composition administered in Step A comprises a monoclonal antibody generated by immunizing mice or other mammals with a conformationally-constrained antigen consisting of amyloid Aβ covalently coupled to colloidal gold via a thioester linkage.

8. A method according to claim 4 wherein the composition is on a surface thereby causing the composition to be conformationally constrained in a shape that corresponds to a conformational-dependent epitope of an aggregate that contributes to the formation or biosynthesis of drusen or drusen-like deposits.

9. A method of claim 8 wherein the surface comprises a surface of a film, particle or sheet.

10. A method according to claim 8 wherein the surface comprises a protein.

11. A method according to claim 10 wherein the protein comprises a beta-pleated sheet.

12. A method according to claim 8 wherein the epitope is bound to the surface.

13. A method according to claim 8 wherein the epitope is chemically bonded to the surface.

14. A method according to claim 13 wherein the chemical bond is a covalent bond.

15. A method according to claim 8 wherein the surface comprises a material selected from the group consisting of gold, zinc, cadmium, tin, titanium, silver, selenium, gallium, indium, arsenic, silicon, mixtures thereof and combinations thereof.

16. A method according to claim 8 wherein the surface is on a gold particle.

17. A method according to claim 8 wherein the surface is on a gold particle contained in a colloidal suspension.

18. A method according to claim 1 wherein the aggregate is a protofibrillar aggregate and has a molecular weight in a range of about 10 kDa to about 100,000,000 kDa.

19. A method according to claim 1 wherein the aggregate comprises from two to twenty subunits.

20. A method according to claim 1 wherein the aggregate is a protofibrillar aggregate comprised of five subunits.

21. A method according to claim 1 wherein the aggregate is a protofibrillar aggregate comprised of eight subunits.

22. A method according to claim 1 wherein amyloid peptide monomers are substantially free of the epitope.

23. A method according to claim 1 wherein drusen or drusen-like deposits have been observed in the patient and the method is carried out to prevent or inhibit the development or pathogenesis of a disease associated with drusen or drusen-like deposits.

24. A method according to claim 23 wherein the disease is a chorioretinal disorder.

25. A method according to claim 24 wherein the disease is macular degeneration.

26. A method according to claim 23 wherein the disease is age related macular degeneration.

27. A method according to claim 23 wherein the disease is congenital stationary night blindness.

28. A method according to claim 23 wherein the disease is membranoproliferative glomerulonephritis type II.

29. A method according to claim 23 wherein the disease is elastosis.

30. A method according to claim 23 wherein the disease is a neurodegenerative disease.

31. A method according to claim 30 wherein the disease is Alzheimer's disease.

32. A method according to claim 1 wherein the composition administered in Step A comprises a monoclonal antibody.

33. A method according to claim 1 wherein the composition administered in Step A comprises a polyclonal antibody.

34. A method according to claim 1 wherein the composition administered in Step A comprises an isolated antibody which binds to a conformation-dependent epitope that is preferentially displayed by oligomeric conformations of Aβ and/or other amyloids that contribute to the formation and/or biosynthesis of drusen or drusen-like deposits.

35. A method according to claim 34 wherein the antibody is effective to reduce the toxicity of a toxic oligomer that contributes to the formation or biosynthesis of drusen or drusen-like deposits.

36. A method according to claim 35 wherein the toxic oligomer has a molecular weight in a range of about 10 kDa to about 100,000,000 kDa.

37. A method for diagnosing a disease or disorder characterized by the formation of amyloid lesions or amyloid matter within the body of a human or animal subject, said method comprising the steps of:
A) providing a labeled antibody that binds to a target oligomer that is present in or contributes to the biosynthesis or formation of amyloid lesions or amyloid matter of interest;
B) administering the labeled antibody to the subject such that it binds to the oligomer; and
C) identifying and/or mapping and/or quantifying locations within the subject's body where the labeled antibody has accumulated.

38. A method according to claim 37 wherein the amyloid lesions or amyloid matter of interest comprise drusen and wherein Step C comprises identifying and/or mapping and/or quantifying any locations in the eye where the labeled antibody has accumulated.

39. A method according to any of claims 37 or 38 wherein Step C comprises performing fluorescence angiography after administration of a fluorophore-labeled antibody

40. A labeled antibody that binds to a target oligomer that is present in or contributes to the biosynthesis or formation of drusen or drusen-like deposits and is useable to perform a method according to claim 37 or 38.

41. A method for delivering a therapeutic or diagnostic agent to a location within the body of a human or animal subject where an amyloid-containing lesion or amyloid-containing matter has formed or potentially will be formed, said method comprising the steps of:
A) providing an antibody that binds to a target oligomer that is present in or contributes to the biosynthesis or formation of amyloid lesions or amyloid matter of interest;
B) crosslinking or otherwise attaching the antibody to the therapeutic or diagnostic agent to form an antibody/agent composition;
C) administering the antibody/agent composition to the subject such that the antibody of the antibody/agent composition becomes bound to the target oligomer within the subject's body.

42. A method according to claim 41 wherein the amyloid lesions or matter of interest comprise drusen.

43. A method according to claim 41 wherein the amyloid lesions or amyloid matter of interest comprise brain lesions, plaques, tangles, firbrils and/or pre-fibril aggregates associated with Alzheimer's disease and/or other amyloid encephalopathies.

44. A method for inhibiting the formation and/or biosynthesis of drusen or drusen-like deposits, or for causing drusen or drusen-like deposits to diminish, in a human or animal subject, said method comprising the step of:
A) administering to the subject, in a therapeutically effective amount, a composition that comprises an amyloid beta-derived diffusible ligand (ADDL) or an antibody that binds to amyloid beta-derived diffusible ligand (ADDL).

45. A method for inhibiting the formation and/or biosynthesis of drusen or drusen-like deposits, or for causing drusen or drusen-like deposits to diminish, in a human or animal subject, said method comprising the step of:
A) administering to the subject, in a therapeutically effective amount, a composition that comprises i) an antibody that binds to an epitope within residues 1-17 of amyloid Aβ and/or ii) a polypeptide that comprises an immunogenic fragment of amyloid A-beta and/or iii) another composition that inhibits the formation of amyloid Aβ.

46. An antibody/agent combination useable to perform the method of claim 45.

47. A method according to any of claims 1-46 wherein the agent comprises a humanized antibody.

48. A method according to any of claims 1-46 wherein the agent comprises a humanized mouse antibody.

49. A method according to any of claims 1-46 wherein the agent comprises a humanized rabbit antibody.

50. A method according to claim 1 wherein the composition comprises a monoclonal antibody generated by immunizing an animal with a conformationally-constrained immunogen consisting of amyloid Aβ covalently coupled to colloidal gold via a thioester linkage and humanizing said antibody.

51. A method according to any of claims 1-46 wherein the agent is delivered directly into the eye.

52. A method according to claim 51 wherein the agent is injected into the eye.

53. A use, in the manufacture of a preparation for administration to a human or animal patient for the performace of the method recited in any of claims 1-52, of a composition that comprises a) a conformational epitope of an aggregate that contributes to the formation or biosynthesis of drusen or the drusen-like deposit and/or b) an antibody that binds to a conformational epitope of an aggregate that contributes to the formation or biosynthesis of drusen or the drusen-like deposit and/or c) a composition that comprises an amyloid beta-derived diffusible ligand (ADDL) and/or d) an antibody that binds to amyloid beta-derived diffusible ligand (ADDL).
